(19) United States
(12) Patent Application Publication
(10) Pub. No.: US 2023/0233722 A1
(43) Pub. Date: Jul. 27, 2023

(54) ARTICLE WITH PATHOGEN INHIBITING TREATMENT

(71) Applicant: SURVIVON LTD., Eveleigh, New South Wales (AU)

(72) Inventors: Thomas HUSSEY, Drumoyne, New South Wales (AU); Scott WHITBY, Chatswood, New South Wales (AU)

(21) Appl. No.: 18/007,930

(22) PCT Filed: Jun. 3, 2021
(86) PCT No.: PCT/AU2021/050555

§ 371 Date: Dec. 2, 2022

(30) Foreign Application Priority Data
Jun. 4, 2020 (AU) 2020901837
Jan. 5, 2021 (AU) 2021900010

(51) Int. Cl. A61L 2/232; A01N 59/20; A01N 59/16; A01N 59/06; A01N 59/00; A01P 1/00; A61L 2/238; D06M 23/06; D06M 11/83; D06M 11/51; D06M 11/46; D06M 11/79; D06M 16/00; A41D 13/11; A41D 31/30

(52) U.S. Cl. CPC A61L 2/232; A01N 59/20; A01N 59/16; A01N 59/06; A01N 59/00; A01P 1/00; A61L 2/238; D06M 23/06; D06M 11/83; D06M 11/51; D06M 11/46; D06M 11/79; D06M 16/00; A41D 13/1192; A41D 31/30; A62B 23/025

(57) ABSTRACT

A substrate with a pathogen inhibiting treatment. The substrate comprising a first coating of an inorganic material. The inorganic material being applied to the substrate via a vapour deposition process. A second coating applied at an upper surface of the first coating, and wherein the second coating is at least one of a protective coating for the first coating and a functional coating.

### TECHNICAL FIELD

The present invention relates to a substrate with at least one of a viral inhibiting coating, a biocidal coating, and a microbial inhibiting coating. More particularly, the present invention may relate to coatings and treatments for inhibiting and/or destroying pathogens.

### BACKGROUND

Viral and biocidal coatings are known in the field of medicine and personal protective industries. Coatings for clothing and surfaces which can inhibit, disrupt, or destroy viruses, microorganisms, microbiological matter, and bacteria can have a wide range of applications and are generally used for high exposure environments and are of particular use during pandemics. There are a number of ways pathogens can be transported and spread, and therefore treatment of surfaces can assist with reducing the potential for transport and spread of pathogens if coatings or anti-pathogen treatments are present.

Airborne viral infection is commonly caused by inhalation of droplets of moisture containing virus particles. Larger virus-containing droplets are deposited in the nose, while smaller droplets or nano particles find their way into the human body. Viruses, which generally have sizes of around 20-500 nm, can be spread by droplets produced by coughing and sneezing.

Masks with smaller fibre gaps will result in breathing difficulty. Other nano-scaled airborne viruses and particles as smoke and super fine dust can enter into human lungs and then into blood system through respiratory membranes. As such, there are a number of issues with further restricting the gaps between fibres to reduce the potential for viral matter to pass through an item of PPE. This is to say that the balance between breathability and filtration and comfort may be difficult to manage.

There are currently two general types of decontamination methods for biological agents: chemical disinfection and physical decontamination. Chemical disinfectants, such as hypochlorite solutions, are useful but are corrosive to most metals and fabrics, as well as to human skin. Physical decontamination typically usually involves using dry heat or super-heated steam for extended periods of time. UV light may also be used but may have variability in effectiveness.

These methods have many drawbacks. The use of chemical disinfectants can be harmful to personnel and equipment due to the corrosiveness and toxicity of the disinfectants. Furthermore, chemical disinfectants result in large quantities of effluent which must be disposed of in an environmentally sound manner. Physical decontamination methods are lacking because they require large expenditures of energy. Both chemical and physical methods are difficult to use directly at the contaminated site due to bulky equipment and/or large quantities of liquids which must be transported to the site. Finally, while a particular decontamination or disinfection method may be suitable for biological decontamination, it is generally not effective against chemical agents. There is a need for decontamination compounds which are effective against a wide variety of both chemical and biological agents, have low energy requirements, are easily transportable, do not harm skin or equipment, and employ small amounts of liquids with minimal or no effluent.

Further the effectiveness and the application of pathogen killing treatments may have a number of varying impacts on an item to receive the coating. For example, the application of films with adhesive are generally undesirable as these films reduce or prevent breathability of an item, which may be critical for wearable items. Further, the use of nanoparticles which may not be securely bonded also have an adverse impact on the environment or the wearer if the nanoparticles were to be dislodged. As such, there may be a need to provide for a pathogen killing treatment which can address the issues of arising from conventional applications.

Any discussion of the prior art throughout the specification should in no way be considered as an admission that such prior art is widely known or forms part of common general knowledge in the field.

### SUMMARY

### Problems To Be Solved

It may be advantageous to provide for a substrate with a viral-inhibiting coating or treatment.

It may be advantageous to provide for a substrate with an oligodynamic property.

It may be advantageous to provide for a which can be functionalised and have an antibiological or antiviral treatment.

It may be advantageous to provide for a treatment method for applying an inhibiting or disruptive treatment to a substrate.

It may be advantageous to provide for a treatment or coating to a medical device to reduce or remove at least one pathogen.

It may be advantageous to provide for a method for applying a pathogen inhibiting or pathogen destroying coating.

It may be advantageous to provide for an inorganic coating which can prevent the residence time of a pathogen on a surface.

It may be advantageous to control the surface morphology of a deposited material on a substrate.

It may be advantageous to control the surface topography of a deposited material on a substrate.

It may be advantageous to provide a substrate with a selective coating.

It may be advantageous to provide a coating to a substrate without reducing the bonding properties of the substrate.

It is an object of the present invention to overcome or ameliorate at least one of the disadvantages of the prior art, or to provide a useful alternative.

### Means for Solving the Problem

In a first aspect there may be provided a substrate with a pathogen inhibiting treatment. The substrate may comprise a first coating of an inorganic material applied to the substrate via a vapour deposition process. A second coating may be at an upper surface of the first coating and wherein the second coating may be at least one of a protective coating for the first coating and a functional coating.

Preferably, the inorganic material may be selected from the following group; titanium, aluminium, zinc, gold, silver, cesium, copper, sulfates of calcium, strontium, barium, zinc sulfide, copper sulfide, titanium dioxide and barium zeolites, brass, mica, talc, kaolin, mullite, lead, mercury, silica and oxides of any of the preceding inorganic materials. Preferably, the first coating may comprise pathogen disruptive ions. Preferably, the second coating may comprise one or more pores to allow the diffusion of ions from the first coating to an exposed surface of the second coating. Preferably, an average surface roughness of the upper surface of the first coating may be in the range of 10 nm to 30 nm Preferably, the average surface roughness of the second coating may be in the range of 10 nm to 50 nm Preferably, the first coating comprises a surface morphology in which grain peaks project from the surface of the substrate at an angle of between 20 degrees to 40 degrees relative to perpendicular angle of the surface. Preferably, the first coating may be a continuous coating of pathogen inhibiting material across at least 80% of the surface of the substrate. Preferably, an interface between the first coating and the second coating may allow for diffusion of ions from the first coating 30 through to an upper surface 60 of the second coating 50. Preferably, the average diameter of at least 80% of the grains of the first coating at the upper surface may be in the range of 5 nm to 50 nm Preferably, the substrate may have a differential pressure of less than 4 mm H₂O/cm².

In another aspect there may be provided an article with a pathogen inhibiting coating. The article may comprise a non-woven substrate and a first coating applied to the non-woven substrate. The first coating may be a pathogen inhibiting layer; and wherein the pathogen inhibiting layer may comprise an inorganic material which forms at least 80% by weight of said pathogen inhibiting layer and may be adapted to release ions to inhibit a pathogen.

Preferably, a second coating may be applied to the article. Preferably, the second coating may be applied to the first coating. Preferably, the inorganic material may be selected from the following group; titanium, aluminium, zinc, gold, silver, cesium, copper, sulfates of calcium, strontium, barium, zinc sulfide, copper sulfide, titanium dioxide and barium zeolites, brass, mica, talc, kaolin, mullite, lead, mercury, silica and oxides of any of the preceding inorganic materials. Preferably, the first coating may have a surface roughness in the range of 5 nm to 50 nm. Preferably, at least one of the substrate and the first coating may be statically charged.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates a side view of an embodiment of a substrate with a pathogen inhibiting treatment;

FIG. 2 illustrates a side view of another embodiment of a substrate with a deposition;

FIG. 3 illustrates a side view of another embodiment of a substrate with a pathogen inhibiting layer with a further oxide layer;

FIG. 4 a side view of another embodiment of a substrate with a coating applied thereto;

FIG. 5 a side view of another embodiment of a substrate with two coatings applied to the substrate;

FIG. 6 illustrates a further side view of another embodiment of a substrate with a coating with angled grain orientation applied thereto;

FIG. 7 illustrates an isometric view of an embodiment of a surface topography of a deposited material;

FIG. 8 illustrates an isometric view of an embodiment of a surface topography of a deposited material;

FIG. 9 illustrates a top view of an AFM image a surface topography of a deposited material;

FIG. 10 illustrates an embodiment of a surface roughness measurement taken of a segment from a sample of substrate;

FIG. 11 illustrates an embodiment of two surface roughness measurements taken from different locations on a surface of a substrate;

FIG. 12 illustrates a sectional view of an example of the surface topography of a substrate with a pathogen inhibiting layer;

FIG. 13 illustrates a sectional view of embodiment of a substrate with a film formed thereon with a peak formation;

FIG. 14A illustrates a perspective view of an embodiment of a roll of substrate with a metal coating applied in a predetermined region;

FIG. 14B illustrates a perspective view of an embodiment of a roll of substrate with a metal coating applied in plurality of predetermined regions;

FIG. 14C illustrates a top view of an embodiment of a substrate with a metal coating applied in a predetermined region

FIG. 15A illustrates an embodiment of a sectional view of an article construction; and

FIG. 15B illustrates another embodiment of a sectional view of yet another article construction.

### DESCRIPTION OF THE INVENTION

Preferred embodiments of the invention will now be described with reference to the accompanying drawings and non-limiting examples.

### REFERENCES

1 Article
10 Substrate
15 Substrate upper surface
20 Substrate lower surface
25 Uncoated region
27 Uncoated region upper surface
30 First coating
35 First coating upper surface
40 First coating lower surface
42 Median upper surface
45 Oxide
50 Second coating
55 Second coating upper surface
60 Second coating lower surface
62 Median upper surface
70 Grains
75 Grain boundary
80 Peaks
85 Outcrop
90 Valleys
95 Minor valleys
100, 100′ Roll
105 Core
110 Roll edge
150 Mask
160 Inner layer
165 Middle layer
170 Fold
180 Boding region
190 Flap
195 Flap bonding surface
197 Flap end

The present invention relates to substrates, which may be primarily used for apparel and garments, and more notably for pathogen inhibiting applications. The substrate 10 is preferably provided with at least a pathogen inhibiting layer, which may be a coating layer which may also provide for self-cleaning. The substrate 10 may be disposed adjacent to one or more additional substrates which can be used to form an article 1. The article 1 may have applications and uses including use for filtration, pathogen inhibition, self-cleaning and personal protective equipment.

The term “pathogen disruptive layer” will be used herein to describe a material which has been deposited by a vacuum vapour deposition method. This material may include any of the inorganic materials mentioned herein and be used to kill, disrupt, inhibit, or otherwise destroy pathogens which come into contact with the deposited surface, or an ion released therefrom, preferably during a period of minutes to hours.

The pathogen inhibiting layer, which may be a coating such as first coating 30 or second coating 50, may be applied using at least one of the following methods: Physical vapour deposition (PVD), plasma enhanced physical vapour deposition (PEPVD), chemical vapour deposition (CVD) and plasma enhanced chemical vapour deposition (PECVD). These processes have many uses in a wide range of industries, notably the automotive, plumbing and food packaging industries. Other industries may also utilise these processes to produce goods which have a chemical coating, in which an article may or may not be pre-treated by a plasma field prior to deposition of a chemical or other element or compound. However, there are a number of problems associated with the above processes in relation to deposition onto textiles, non-wovens and other fibrous substrates.

A PVD process typically uses a PVD source material for deposition onto an article. In a PVD or PEPVD processes, PVD source material is evaporated via evaporation processes and the evaporated material will then condense on the article (evaporation PVD) to create the desired deposition, or a sputtering process will displace a source material to be deposited and condense on an article (sputtering PVD). For PVD processing, there are no chemical reactions that take place in the entire process, unlike CVD processes. As there are no chemical reactions, the purity of the source material may be required to be of a high purity, which may limit the conditions in which the PVD can occur.

Notably, bonding strength between the deposition and the treated substrate 10 or consistency and quality of the coating may also be improved and result in a superior product from vapour deposition processes. Bonding between nanoparticles or pathogen inhibiting materials and the substrate 10 are generally concerns for pathogen inhibiting substrates known in the art. As such, the present disclosure may provide a clear advantage over the known state of the art.

Suitable substrates 10 which can be coated or deposited with a coating which may include a film, a textile, a fabric, or any other desired generally planar surface. The pathogen inhibiting layer may be a first coating 30 or second coating 50, for example. The second coating 50 may be formed from any desired material described herein, and may be selected from any material which the first coating may be formed from. While it is preferred that a planar surface is treated with a coating, the coating can be applied to irregular or textured surfaces or three-dimensional objects such as electronics or peripherals therefor. More than one coating may be applied to a surface of an object or substrate 10 to create a desired functional or pathogen inhibiting treatment. The combination of a substrate or object with a coating may be referred to herein as an “article” 1.

A textile or fabric may include at least one of; nylon, polyamide, rayon, polyester, PP, PET, PE, aramid, acrylic, acrylate, paper, wool, silk, cotton, linen, Kevlar®, lyocell (Tencell®), fibre glass, glass, woven textiles, non-woven textiles, knitted textiles, braided textiles, insulation materials, synthetic materials and fibres, natural materials and fibres, organic materials or any other material which may be suitable for use in a garment, PPE, face mask, filter, drapes, bedding, wall covering, and upholstered products. It will be appreciated that a textile is a substrate 10 formed with yarns, filaments, strands or fibres which are interconnected in a regular or ordered manner (woven or knitted textiles) or bonded together in the case of non-woven textiles.

These textiles have pores or gaps between fibres, yarns, filaments, or strands, which makes these textiles breathable which is a highly desired property for garments, and for a number of filtration devices and mediums.

Gaps and pores of a textile may also increase the overall surface area of a side of the textile and therefore pathogen inhibiting layers applied thereto may also have a generally larger surface area which may be advantageous for catching, or inhibiting pathogens. However, the pathogen inhibiting layer may also be formed such that the overall surface area is increased or has at least one texture to increase the surface area compared to more effectively inhibit pathogens. A pathogen inhibiting layer may have variable thicknesses to allow for different pathogen inhibiting applications or periods of pathogen inhibiting potential. For example, a relatively thicker pathogen inhibiting layer may allow for a longer period of pathogen disruption compared to that of thinner pathogen inhibiting layers or compared to that of conventional coatings which include dispersed nanoparticles.

The substrate 10 may have a first coating 30, which is a pathogen inhibiting layer, conferring antimicrobial properties or pathogen inhibiting properties. The first coating may be selected from one or more of the following group; titanium, aluminium, zinc, gold, silver, cesium, copper, sulfates of calcium, strontium, barium, zinc sulfide, copper sulfide, titanium dioxide and barium zeolites, brass, mica, talc, kaolin, mullite or silica and oxides of any of the preceding inorganic materials. In addition, lead or mercury compounds may also have some use depending on the application. As the pathogen inhibiting layer is formed by a condensation of vapour, an average diameter or cross-sectional size of the metal ‘islands’ or peaks formed by the deposition at the surface of the deposition film may be between 10 nm and 200 nm, preferably in the range 5 nm to 100 nm Further, the overall thickness of the deposited film on the substrate may be in the range of 5 nm to 200 nm, although thicker coatings can be applied if desired depending on the speed of the substrate and vapour cloud density during deposition. It will be appreciated that the pathogen inhibiting material may be a near pure metal (greater than 95% purity, or more preferably greater than 99% purity), a metal alloy, or sulfides or sulfates of any other aforementioned metals.

A significant advantage of utilising vapour deposition processes to form a pathogen inhibiting layer is that the pathogen inhibiting layer can be formed with any desired thickness, and therefore a relatively large number of ions can be released from the pathogen inhibiting layer or there is a greater potential for ions to be released from said pathogen inhibiting layer, relative to that of traditional methods of applying an ion releasing coating. Traditional ion releasing coatings are generally applied with a padding process, spray coating other wet coating process whereby a liqor is applied to the substrate and then dried and/or cured to form a coating. These methods rely heavily on the ability of the liqor to wet the surface of substrate during the coating process. As such, if the substrate is of a relatively thin structure, the total amount of functional coating material which can be applied to the substrate 10 is reduced compared to thicker substrates. Further, if the substrate has hydrophobic properties or a hydrophobic functional coating, the amount of ion releasing material which can be applied to the substrate 10 is again reduced. For example, polypropylene may be readily statically charged which is advantageous for some applications, however the wettability of polypropylene is low and therefore exhibits a level of hydrophobicity and therefore is difficult to coat with a functional coating or a coating with an ion releasing particle. In stark contrast, the application of a film using PVD or CVD processes overcomes the limitations inherent with conventional application methods. In addition, the use of conventional nanoparticle solutions cannot achieve a continuous, or semi-continuous film of ion releasing material and instead relies on nanoparticles dispersed throughout the resin or coating suspension.

It is preferred that the pathogen inhibiting layer has a relatively low solubility in aqueous media. Optionally, the pathogen inhibiting layer may also be an alloy of silver with copper or zinc. Optionally the alloy may be at least one of a copper alloy, copper oxide, zinc alloy, and zinc oxide. The pathogen inhibiting layer is adapted to release ions at an effective level for pathogen disruption or antimicrobial activity over a prolonged period, such as months or preferably years. Preferred metals may be silver, copper, or zinc as these are generally readily available materials which have minimal potential adverse effects for use in medical devices or personal protective equipment (PPE). Components which meet these criteria are silver, silver oxide, silver halides, copper, copper (I) oxide, copper (II) oxide, copper sulfide, zinc oxide, zinc sulfide, zinc silicate and mixtures thereof. Mixtures of silver with zinc silicate and silver with copper (II) oxide are preferred. Optionally, the first coating and/or the second coating may instead be a self-cleaning or oligodynamic treatment such as a TiO₂ coating or the like. Other self-cleaning materials are well known in the art and may be selected based on the desired end function of the substrate 10.

Optionally inorganic salts may be used to assist with pathogen inhibition. The term “inorganic salt” includes inorganic metal compounds that are relatively insoluble in water. Inorganic salts may include ionic metal compounds whose cations together with anions of other inorganic substances form a compound. When such salts are placed near water, these compounds usually release ions. Salts with low water solubility, i.e. less than 100 mg/L and less than 15 mg/L may be desirable for use with at least one nanoparticle coating.

The term “cation release” generally refers to providing a cation from a metal salt suspended by a functionalizing agent to the environment in which a pathogen is located. This release mechanism is not a controlled feature of the present invention. In one embodiment, the release occurs, for example, when ions dissolve from inorganic particles. Any number of mechanisms can cause the release of cations, and the article may include any desired mechanism for release of said ions.

In a preferred embodiment the pathogen inhibiting layer includes copper and/or silver as a pathogen inhibiting material. Silver and copper have been observed to have a trace effect on bacteria with the silver ions and copper ions being used to denature proteins in target bacteria by binding to reactive groups. This binding can result in precipitation and deactivation of a pathogen. Silver has also been shown to inhibit enzymes and metabolic processes. Cationic species are electrostatically attracted to the negatively charged bacterial cell walls. Cationic antimicrobial peptides have been shown to have an inhibitory effect on target bacterial regulatory mechanisms. As such, the substrate and/or the coatings applied thereto may be statically charged to encourage particle to fabric attractions, for example attracting a pathogen or virus toward a substrate.

While silver and copper have been shown to provide such denaturing effects, other inorganic materials may also have a number of benefits which can be used for self-cleaning, self-sterilisation, biocidal, pathogen inhibiting, pathogen-killing or oligodynamic effects.

The pathogen inhibiting layer may be formed with at least 50% by weight pathogen inhibiting material. Optionally, the substrate 10 may have a precoating or primer formed with at least 1% to 4% by weight alumina which can promote pathogen inhibiting properties conferred by the first coating.

The second protective coating may be formed on the surface of the first coating or may be applied to the first coating in the case of a film or membrane. The second coating may be selected from silica, silicates, borosilicates, aluminosilicates, alumina, aluminium phosphate, or mixtures thereof. The second coating functions as a barrier between the antimicrobial particle and may assist with the rate of diffusion of the pathogen inhibiting layer. Preferably, the second coating can also be a functional coating which can provide one or more desired functional properties.

The functional properties of the second coating may include at least one property from the following group; flame retardancy, abrasion resistance UV absorption, self-leaning, hydrophobic, hydrophilic, and/or antibacterial properties.

While the second coating is applied to the first coating, the second coating allows for the diffusion of ions through the second layer which provides the pathogen inhibiting properties for the substrate 10. The second coating may also include nanoparticles or material used to form the first coating. This may assist with diffusion of ions from the first coating to the upper surface of the second coating. To assist with diffusion, the second coating may be formed with a porous structure which allows the movement of ions, while reducing or prohibiting the ingress of fluids or other foreign matter into the pores or gaps within the second coating. It will be appreciated that if the first and/or second coatings are applied to a woven or non-woven material which includes gaps or pores, fluids such as oxygen or ambient atmosphere may be allowed to pass through the substrate via these gaps or pores, and should not be confused with the gaps or pores formed within the second coating.

The second protective coating layer may include a pathogen inhibiting material similar to that of the first coating, but preferably includes dispersed nanoparticles in the range of 5 nm to 200 nm in size, which can improve the pathogen inhibiting ability of the substrate. The second coating may have the nanoparticles encapsulated within the matrix of the second coating, which may be of benefit if the second coating is a functional coating. If a second coating is applied, the surface coverage of the first coating by the second coating is preferably at least 80%, but is preferably at least 90%. It is preferred that the second layer formed has pores to assist with diffusion of the pathogen inhibiting layer to allow metal ions through the second coating at a slow rate, while functioning as a barrier which limits interaction between the pathogen inhibiting layer and foreign objects.

Utilising the first coating may provide a reduction in infectious viral titre of fabric exposed to a virus. After exposure of the fabric to known concentration of virus for a period of 30 minutes or greater, the infectious virus titre may be reduced by at least 99.9% compared to the infectious viral titre of the original inoculum. More preferably, the reduction of infectious virus titre is at least 99.9%, or greater than 99.99% or at least 99.999%. The present invention may provide a means for reducing viral activity on a fabric such that infection or transmission of a virus is prevented or avoided to a significant extent. More preferably, there is observed a log 3 reduction of viral activity.

In one embodiment, the first coating may provide for a means to reduce the persistence of a virus on the first coating or a coating adjacent to the first coating. Preferably the virus is any virus from the following group consisting of; Influenza, Measles, SARS-CoV, SARS-CoV-2, MERS-CoV, Coronavirus, Mumps, Marburg, Ebola, Rubella, Rhinovirus, Poliovirus, Hepatitis A, Smallpox, Chicken-pox, Severe Acute Respiratory Syndrome virus or SARS virus (also referred to as SARS coronavirus), Human Immunodeficiency Virus (HIV) and associated non-human animal immunodeficiency retroviruses such as Simian Immunodeficiency Virus (SIV), Rotavirus, Norwalk virus and Adenovirus. Norwalk virus includes its surrogate Feline Calicivirus. Influenza viruses include both human and avian forms of the virus.

Optionally, one or more primer coatings may be applied to a substrate in advance of application of a first coating 30 to the substrate 10. A primed substrate may provide for a smoother surface for a first coating 30 to be applied thereto, or the primed substrate 10 may be used to improve the bond strength between a coating and the substrate. Having a smoother substrate may allow for a more uniform application of a first coating, which can improve the pathogen inhibiting properties when in use. Primers may be applied using CVD or PVD techniques depending on the substrate and/or the pathogen inhibiting layer and/or coating layer. Primers may be used to increase or decrease the porosity of the substrate 10 and may also be used to bond or adhere adjacent fibres of the substrate. Any primer used may also be used as a chemical barrier which may assist with waterproofing, breathability or another functional property for the substrate. It will be appreciated that any substrate 10 may be primed on one or both sides of the substrate but will usually be at least the side to be bonded with or fixed with a first coating 30. Optionally, the first coating 30 may act as a primer for a further coating, such as the second coating 50.

While it is preferred that the first coating 30 is applied with a vapour deposition process, the second coating 50 may also be applied using a vapour deposition process or a conventional spray or padding process.

Preferably the first coating 30 may be deposited in a pure elemental form (purity greater than 97%), the first coating 30 may be optionally exposed to oxygen or another reactive element or compound to oxidise the first coating 30 to form an oxide layer 45 or convert a portion of the first coating 30 into a salt. Oxidation of the deposition may occur near to the surface of the first coating 30 and/or at the interface between the substrate 10 and the first coating 30 depending on the porosity of the substrate 10. Optionally, any coating or layer formed with by vapour deposition can have an oxide layer or crust formed at at least one of the upper and lower surfaces. The oxidation of the first coating 30 may form a natural protective coating which can assist with maintaining integrity of the deposition which can preferably function as a pathogen inhibiting coating. In some embodiments it may be advantageous to limit the exposure of the first coating 30 to local atmosphere to reduce oxides forming; however, this is optional as diffusion of the first coating may still possible without limiting oxidation.

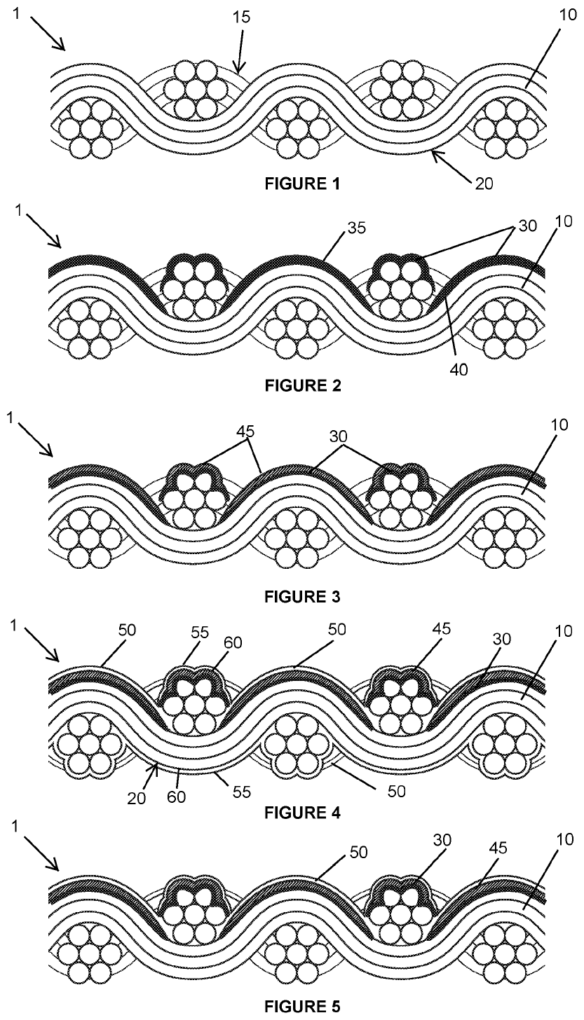

Referring to FIG. 1 there is illustrated an embodiment of an article 1 which includes a substrate 10 with a first coating 30 and a second coating 50 applied thereto. At least one of the first coating 30 and the second coating 50 being a vapour deposition coating. In some embodiments a primer or another bonding coating may be used as a base for a deposition to be applied to such that improved adhesion between the substrate 10 and the coating can be achieved.

FIG. 2 illustrates the substrate of FIG. 1 in which a coating has been applied to the upper surface 15 of the substrate 10. The coating may be a pathogen inhibiting layer and may also be the first coating. It is preferred that the pathogen inhibiting layer is also the first coating, but may be any desired coating of the article 1. For example, if the article 1 comprises five coatings, the pathogen inhibiting layer can be any one or more of the five coatings applied thereto.

In an unillustrated example, a primer may be applied to a surface 15, 20 of the substrate 10 which may be used to improve the adhesion of the first coating 30. The first coating 30 is preferably applied using vapour deposition processes and may have a thickness in the range of 5 nm to 200 nm which is dependent on the speed of the substrate 10 when the first coating 30 is applied, or depending on the density and proximity of a vapour cloud which is used to deposit the first coating 30.

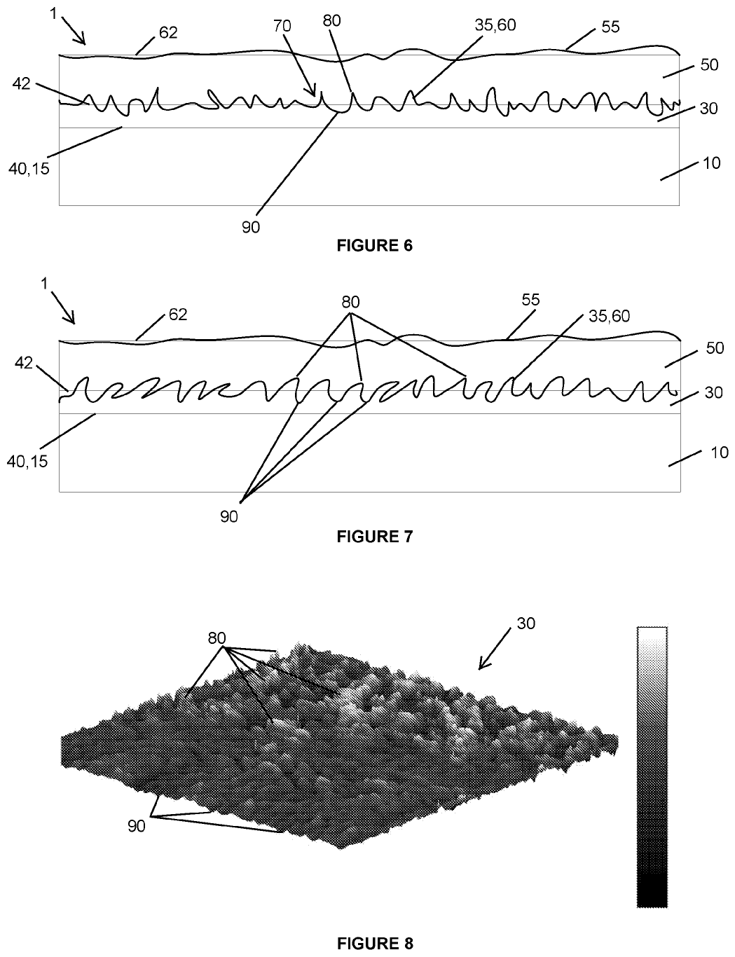

The temperature and the rate of condensation of vapour deposited coatings may also impart one or more grain structures or surface topographies or morphologies to the first coating. Each of grain structures, grain sizes, grain orientation, surface morphology and surface topography may impart a number of properties to a deposited coating, and more particularly may assist with controlling the rate of diffusion or promoting a pathogen inhibiting coating. The number of peaks 80 which are formed, and the orientation of the peaks 80 may be relative to the relative location of the vapour cloud and the velocity of the substrate. For example, if the substrate 10 is transported over a cooling drum, which can both assist with condensation and also limit exposure of a substrate to high radiant temperatures from evaporation sources, the cooling drum may cause vapour to be deposited at an angle which can create surface peaks 80 at an angle rather than surface peaks 80 which are generally perpendicular to the surface of the substrate. An example of these formations are illustrated in FIGS. 6 to 8.

FIG. 3 illustrates a first coating with an oxidation forming at the upper surface of the first coating. The oxide forming at the surface may be either chemically promoted with an oxidation process, etching process or a texturization process. Alternatively, the coating may be formed when the first coating is exposed to atmosphere. The oxide layer as shown is formed at the upper surface of the first coating, but it will be appreciated that the oxide may form where atmosphere can interact with the first coating, and therefore may occur on both upper and lower surfaces of the first coating. This may create an upper and a lower crust on the first coating, and may protect the first coating. Oxides forming at the surface will still preferably allow for diffusion of ions and thereby still allow for pathogen inhibiting properties to be imparted to the article 1. Oxide layers may be a protective coating, or protective layer which protects the first coating, or another coating applied to a substrate 10, or applied to another coating, from abrasion or further oxidation.

Alternatively, the first coating 30 includes metallic nanoparticles which are embedded within a polymeric matrix on alloy which is corrosion resistant or be benign to corrosion (such as gold) and as such the oxide layer may not form at the surfaces of the first coating without more reactive species being introduced to encourage oxidation or corrosion of the surface. Even if the material of the first coating embeds inorganic material which can release ions to inhibit pathogens, the ions from the inorganic material can still be allowed to diffuse to the upper and/or lower surfaces of the first coating.

FIG. 4 illustrates a substrate with a first coating which has a further coating which may be a second coating. The second coating can be seen as coating both the lower surface of the substrate 10 and also the upper surface of the first coating. This coating may be applied with traditional padding processes or other conventional coating processes which can be used to apply a treatment or a functional coating to exposed surfaces of the substrate and any other coatings thereon.

It will be appreciated that a second coating may be the same as, or similar to the first coating, in that metallic particles may be embedded within a polymeric matrix or polymer. An oxide layer may not be formed if the metal is encapsulated or deprived of a reactant to corrode, such as oxygen or water. Alternatively, the second layer 50 may be applied to a primer or other coating, such as a first coating, which already exists on the substrate 10. This may be of particular advantage as the first layer may be a functional coating, such as a hydrophobic, hydrophilic, biocidal coating or pathogen inhibiting coating which may be present at the surface of the substrate 10 before application of the second coating, which may be a metal coating or pathogen disruptive layer. The second coating may then be allowed to oxidise, either by exposure to the atmosphere or by reacting with the first coating. The oxidation of the second layer may be encouraged by the primer or functional coating, such that controlled or desired oxidation can be affected. In another embodiment, the second layer may be protected to reduce or control oxidation. In yet another embodiment the first coating is a metal layer, alloy thereof and the second layer is a protective coating which protects the first coating from oxidation or limits the amount of oxidation of the first coating. In yet another embodiment, a primer may be applied to the substrate 10, a first coating may then be applied to the primer, and a second coating 50 may be applied to the first coating 30, wherein the first coating 30 is a metal or metal alloy and the second coating is a protective coating, such that the primer and the second coating encapsulate or protect the first coating from oxidation, or limit the potential for oxidation.

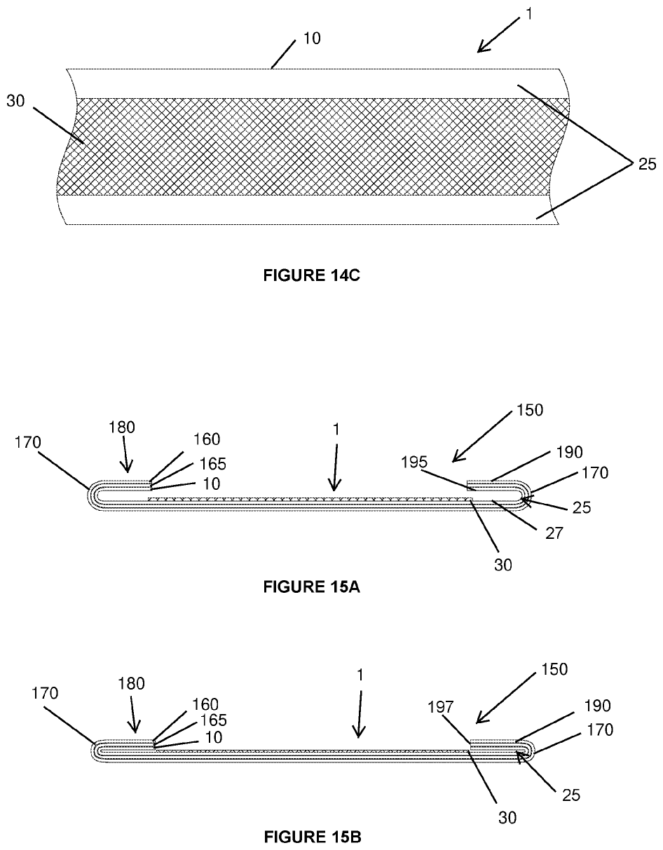

Selective patterns or coatings may be applied to the surface of the metal coating or pathogen disruptive layer, such that predetermined portions or areas of the mask may be oxidised, while other portions are not. Embodiments of a substrate 10 which has received a patterned or selective coating can be seen in FIGS. 14A to 14C. Further, a stencil or other selective coating may be applied to a substrate 10 which leaves a portion of a surface of the substrate 10 uncoated or untreated, or leaves a first coating 30 surface partially uncoated by a second coating 50. This may be of particular advantage as instructions, templates, or silhouettes may be imparted to the mask and defined by regions which are oxidised and other regions which are not. Alternatively, the one or more regions which comprise a different level of oxidation may be imparted to the article 1, which can provide for a visual effect. For example, copper coatings may be dulled, discoloured, colour-altered, or greened when exposed to different levels of oxidation. The oxidation or colour change may be imparted with the use of a laser, roller, hot air, or other predetermined heating or radiation treatment. In this way local discolouration or alterations may be imparted to a relatively small area of a substrate 10. This can be used for branding, marking, and aesthetic purposes.

In yet another embodiment, the second layer is applied to a first layer (which be a metal layer) and the second layer is a reactant or contains reactant particles such as metal particles which may affect a reaction at the interface between the first layer and the second layer. For example, silver particles may be included with the second layer to encourage oxidation of a first layer which may be comprised of a more reactive metal, such as copper. Other materials and metals may be used, and copper and silver are exemplary only. Having the first and second layer interact in this way may improve the bonding between the first and second layers, while also improving the efficacy of the metal layer or pathogen disruptive layer. Efficacy may be improved as corrosion of the metal layer may allow for a greater number of ions to be released and thereby improve the pathogen inhibiting or biocidal properties of the first layer. This may be of particular advantage if the second layer is a hydrophilic layer which may improve the transport of moisture, and pathogens or bacteria therein, to the interface between the first and second layers. This may allow for capture and more effective sterilisation or neutralisation of pathogens or bacteria.

The second layer may alternatively be adapted to increase the moisture or oxygen near to the interface between the first and second layers, and thereby encourage further oxidation until a crust has formed. This crust may be protected by the second layer, while still being adapted to release or diffuse ions to inhibit or kill a pathogen or bacteria interacting with the article 1.

In another embodiment, the article 1 may be adapted to absorb moisture and change colour when in use to signify when the article should be changed. For example, if the article 1 is a face mask, the regions which are to generally cover the mouth and/or nose of the wearer may be less oxidised when first used, but with a period of use may discolour or otherwise oxidise to indicate to the wearer that the mask is ready to be changed for a new mask.

It will be appreciated that any layer on an article 1 may be adapted to function as a first layer as described herein, while not necessarily being the first layer applied to a substrate. Optionally, one or more layers may be adapted to function as the first layer, but instead be the at least one of a second, third or fourth layer in some embodiments (not illustrated).

FIG. 5 illustrates an embodiment similar to that of FIG. 4, however the second coating has been applied to only the first coating. This second coating may be a film or a further deposition coating. The second coating may also be applied by knife coating methods or other similar methods known in the art. The second coating may have at least one functional property, or may be used to assist with protection of the first coating or limiting the diffusion rate of the first coating.

FIG. 6 illustrates an embodiment of the surface appearance of a first coating and a second coating 50 applied to the first coating 30. The first coating may also be a pathogen inhibiting layer and/or the second coating be a pathogen inhibiting layer. The upper surface 35 of the first coating comprises a number of discrete peaks 80 and troughs (valleys 90) formed during the condensation of vapour to form the coating 30. The median upper surface height 42, 62, of the first 30 and second 50 coatings respectively, are illustrated as a horizontal line which is considered to be the surface level. The peaks 80 project from the surface level and the valleys 90 extend below the surface level. Similarly, the second coating 50 is shown with a surface morphology which is different to that of the first coating 30. The morphology of the second coating may be different due to a different process used to apply the coating 50, or may be due to a different composition of the second coating, such as whether there is an organic or inorganic coating formed. However, regardless of the method of application of the second coating, the first coating and the second coating may have different surface morphologies at their respective upper surfaces 35, 55. The interface between the first coating 30 and the second coating 50 is illustrated as being fully bonded, however micropores or microgaps may be present at the interface between the two coatings. Pores and gaps in the structure of the second coating 50 can be used to assist with ion diffusion from the first coating 30 to the upper surface 55. Preferably, the surface of the second coating 50 has a lower surface roughness compared to that of the first coating 30. Although, for some functionalisation coatings this may not be desired.

As can be seen the formations 80, 90 at the upper surface 35 of the first coating are generally vertical with some formations having minor non-uniform angling. The peaks 80 may be locations which promote diffusion of ions which can progress through the any additional coatings applied thereto, such as the illustrated second coating 50.

FIG. 7 illustrates the surface morphology which can be imparted by a vapour cloud which is offset from a coating drum. As shown the peak formations projecting from the surface of the first coating are generally conforming to an angle between 15 to 30 degrees form the perpendicular angle relative to the surface. While the illustrative embodiment shows variation of the peak 80 formations, in practice the formations may be more regular and more evenly spaced depending on the vapour cloud, the vaporisation temperature, the condensation rate and the speed of the substrate 10.

FIG. 8 illustrates the surface topography and morphology of a sample of substrate which has received a vapour deposition coating. The surface topography shown includes a number of peaks 80 which project from the surface and have been imparted with an angled orientation or angled projection. The angled nature of the grains of the deposited film may be imparted having the coating of the substrate occur on a curved surface with a vapour cloud disposed relatively under the curvature such that the vapour moves upwardly to contact the substrate 10. The curved nature of the deposition may be desired for a number of applications and may also assist with reducing the thickness of the deposition and increasing the surface area at the upper surface which can assist with pathogen inhibiting properties.

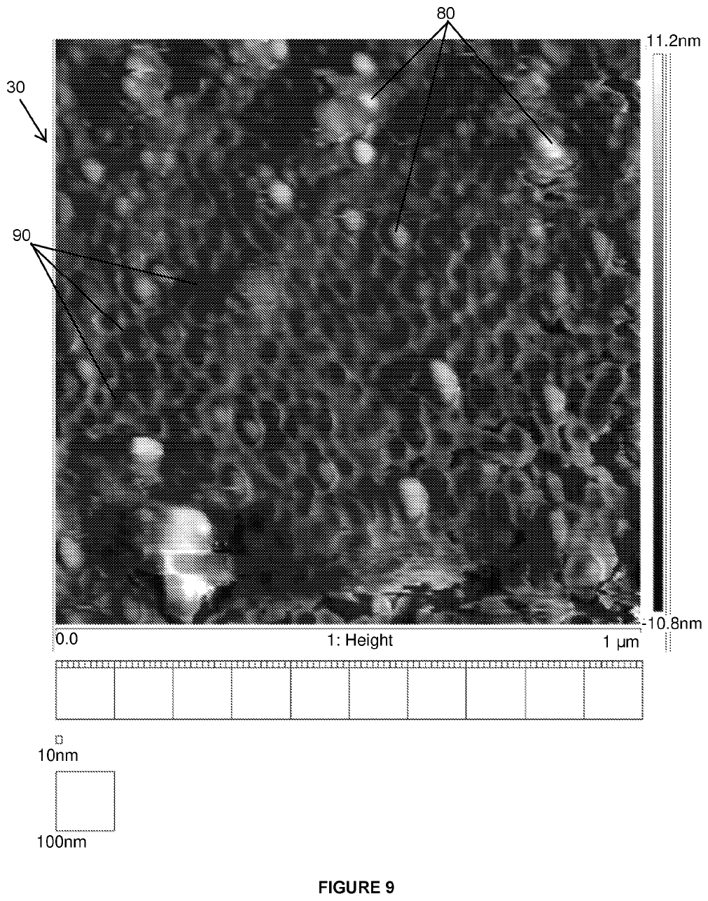

FIG. 9 depicts a top view of a film deposition on a substrate 10. The grain structures 70 form peaks 80 at the upper surface of the coating and are generally circular with few polygonal grains being formed. The average diameter of the discrete peaks 80 at the surface are around 50 nm. However, it is noted that larger peaks 80 are also present which have larger axis lengths in the range of 70 nm to 200 nm. The larger the peak 80 diameter the greater the potential for the peak to allow for higher diffusion rates. Preferably, the general surface roughness is uniform or regular across larger sample sections of the coated substrate 10. The side of the peaks 80 may have a general slope of between 20° to 80° relative to the surface of the coating.

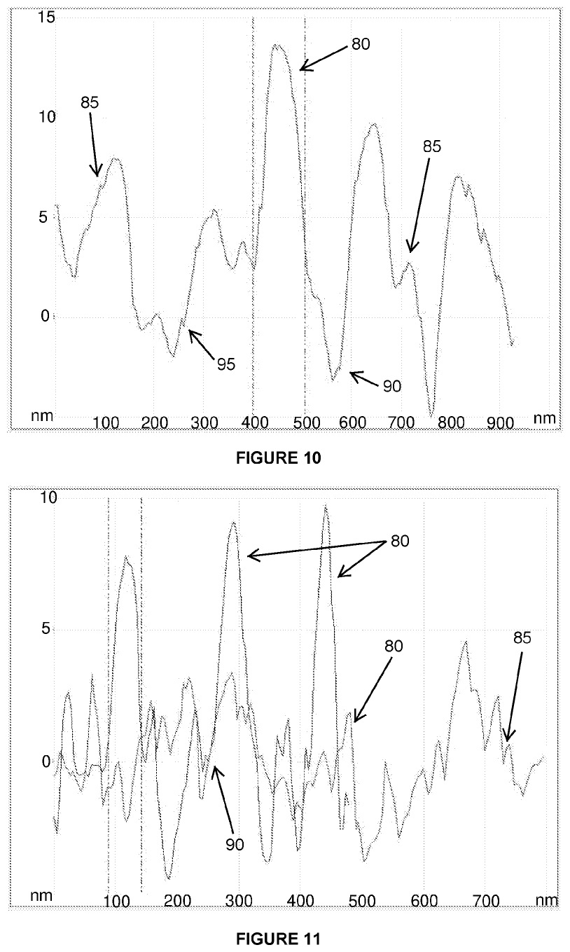

Referring to FIG. 10, there is provided a cross-sectional view of a portion of a surface topography of a coating imparted by a vapour deposition process. The section is representative of an approximate 950 nm topography length with peaks 80 in the range of around 5 nm to 15 nm in height, with valleys around 2 nm to 5 nm in depth. As can be seen, peaks 80 may be formed with outcrops 85 on the sides of the peaks 80. The outcrops 85 may generally be around 0.1 nm to 5 nm in height and may provide for a region of ion diffusion or release of ions. In other embodiment, the height of outcrops 85 may be greater than 5 nm. Further, the outcrops can assist with increasing the overall surface area of the peaks 80 and may further improve the pathogen inhibiting properties of the coating. In another embodiment, outcrops may be any formation on a peak 80 which is less than 5 nm in height and any formations greater than 5 nm are considered to be peaks. It will be appreciated that the size of an outcrop may be dependent on the height of the peaks, and therefore may be considered to be any formation which is less than 10% to 30% of the height of a peak 80. Optionally, peaks 80 may be referred to as major peaks, and outcrops 85 may be referred to as minor peaks. Conversely, valleys 90 may be referred to as major valleys and minor valleys 95 may be defined by the depression between adjacent peaks 80, 85.

Referring to FIG. 11, there is illustrated a further embodiment of a cross sectional profile of a coating surface. The coating surface is a first coating 30 and is a pathogen inhibiting layer. The profile shown comprises sections from two portions of the surface of the coating. A first section is approximately 500 nm in length and a second section is approximately 800 nm in length. Each section represents a change of surface topography such that portions of the surface comprise regions with a higher surface roughness compared to other portions of the surface.

The first section comprises peaks which are formed with heights of up to 10 nm and valleys 90 extending to a depth of around 4 nm to 5 nm. Measuring a peak 80 diameter or cross-sectional area can be determined by the spacing between valleys 90. Peaks 80 of the first section are in the range of around 30 nm to 180 nm and the peaks of the second section are in the range of around 10 nm to 60 nm.

In further embodiments, the surface topography may have at least two major regions which define larger peaks and smaller peaks respectively. Optionally, the coating surface may be formed with transitional regions between the major regions such that a gradient of peak heights is formed there between. Larger peak regions may have a greater pathogen inhibiting ability relative to smaller peak regions.

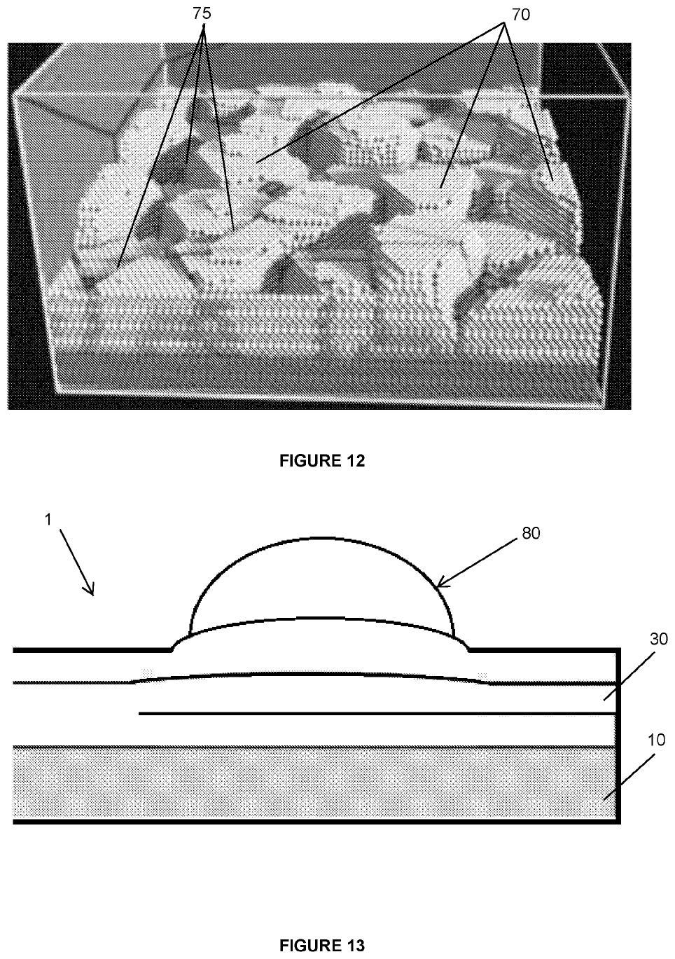

Referring to FIG. 12, there is illustrated an embodiment of a surface topography of a film formed on a substrate. The film includes discrete structures grown via a Stranski-Krastanow mode growth. A continuous layer has been formed with peaks at the surface of the film. The grains 70 are of differing heights with grain boundaries illustrated as generally geometrical, however the grain boundaries 75 may be more rounded depending on the deposition rate, cooling rate and the temperature of the vapour cloud interacting with the substrate 10. The tops of the grains 70 are generally plateau-like in appearance, but may also be formed with rounded tops. Gaps may also be present between the grains 70 which form as the grains grow from the surface during deposition. As can be seen, layers of condensation are illustrated, however these layers may or may not be of uniform thickness and may include defects or abnormal lattice structures which promote the growth of the grains and/or peaks from the surface of the substrate 10.

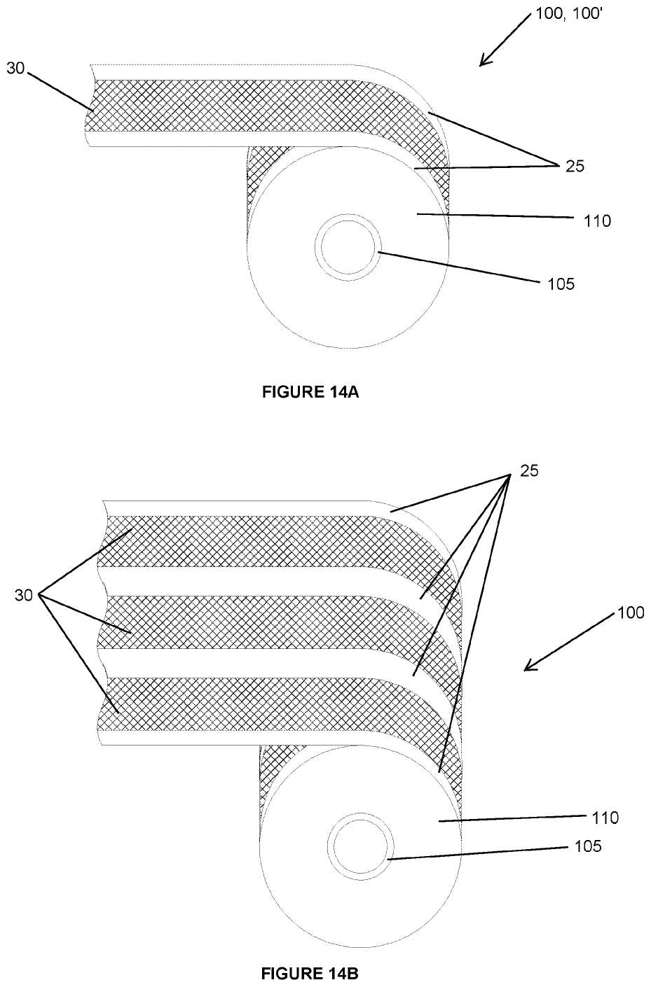

FIG. 13 shows a cross-sectional view of a representation of a peak forming on a substrate. The substrate is a linear substrate 10 with a first coating 30, or film, formed on the surface of the substrate 10. A peak is also formed at the upper surface of the first coating 30 which has been formed as vapour condenses in a localised region. The layer directly below the peak 80 has been formed as a deformation of the film has raised a portion of the film and caused additional deposition material to build up and forma peak on the surface of the film. While it is not illustrated, the film may also have one or more valleys formed which extend below the mean surface level of the film.

Pores or gaps of the substrate 10 are essentially free of blockage or has less than a 5% pore or gap size reduction, or has less than a 5% pore or gap size reduction, or has less than a 4% pore or gap size reduction, or has less than a 3% pore or gap size reduction, or has less than a 2% pore or gap size reduction, or has less than a 1% pore or gap size reduction, after application of the pathogen disruptive layer. This is a significant benefit over common methods of applying a biocide or micro-organism destructive coating which may include the application of a non-permeable film, a padded coating, an adhesive or another permeability reducing coating.

At least one coating may be applied to the substrate 10 by the use of one of the following methods; chemical vapour deposition, physical vapour deposition, sol gel deposition, or a combination thereof.

Thickness of the pathogen inhibiting layer can be in a nano-range and is preferably in the range of 5 nm to 100 nm thick. By coating the fabrics with a pathogen inhibiting layer, the use of biocidal nanoparticles can be avoided and therefore may provide pathogen inhibiting means while also providing for a leach-free coating.

The utility of the pathogen inhibiting layer applied by vapour deposition as above will avoid the use of nanoparticles or biocidal additive chemicals, which could leach into environments or contaminate the skin of a wearer. Generally, the pathogen inhibiting layer used herein is not soluble even further reducing the potential for nanoparticles to be introduced into the environment from the substrate.

Application of a pathogen disruptive layer onto a substrate 10 may provide for an enhanced pathogen inhibiting substrate. Further, the combination of a pathogen inhibiting layer textile and pathogen retaining or dirt/chemical retaining filter medium offers various functions including, but not limited to; pathogen control, chemical control, and dirt control while maintaining a low pressure drop and a high water flow rate when in use. This may be of particular use for air filtration, water purification and filtration, and other fluid capture and cleaning applications. This may also be of particular use in laboratory extraction systems and for personal protective equipment, face masks and clothing.

The filter medium may be any substrate which can filter, retain or capture particles which move into the same plane as the filter medium. The filter medium may be charged with a positive or negative charge to attract particles of the opposite charge. Additionally, the filter medium may be a non-woven material or a generally porous material which can allow desired fluids to pass while capturing pollutants or other particulates. A pathogen inhibiting layer can be applied to the filter medium directly or may be combined with the filter medium to form an article 1. It will be appreciated that the article 1, in some embodiments, may be a filter medium with at least one coating applied thereto, wherein a applied coating is a pathogen inhibiting layer.

The combination of a pathogen inhibiting layer and a filter medium may be desired as the filter medium can be used to capture and retain the pathogen to allow for an effective time period for the pathogen inhibiting layer to kill, disrupt, inhibit, or otherwise destroy pathogens captured. In addition, it is preferred that the gaps between fibres or the pore size of the filter material is as large as possible for some applications, such as for filtration masks, to allow a desired breathability. However, having a larger gap or pore size may reduce the potential to physically capture particulates and therefore charged fibres may be used to draw particulates to the filter medium and retain the particulates.

In another embodiment, there may be provided a filter substrate or filter medium. The filter medium may comprise a silver or copper coated textile with at least one functional treatment applied thereto. It will be appreciated that any of the pathogen inhibiting depositions mentioned herein may be applied to a filter medium. Optionally, a membrane may be included which can be used to filter and/or capture dirt, and/or a chemical-retaining membrane, and/or pathogen-retaining membrane, or a combination thereof. The membrane can be disposed between the substrate and a coating, such as that the coating is applied directly to a membrane. The membrane may be integrally formed with substrate 10 or may be fixed or adhered to the substrate 10.

In yet a further embodiment, the substrate 10 may be a protective barrier which may assist with reducing or prevent the penetration of a pathogen through said substrate 10. These substrates 10 may preferably allow moisture to pass through the substrate in at least one direction. Non-wovens with moisture vapor permeable membranes such as a microporous PE membrane may be suitable for use as a substrate 10, or a part thereof.

Further, the invention, in at least a preferred embodiment, relates to the use of textiles which are coated with a silver deposition (or other biocidal material). The textile can be used for forming a pathogen-retaining filter medium, for providing filter medium with enhanced pathogen killing efficacy and for pathogen disruptive or pathogen inhibiting protective equipment.

Accordingly, a preferred embodiment of the invention provides filter medium comprising a microorganism-killing membrane. The microorganism-killing membrane includes a textile deposited with at least one pathogen or microorganism disruptive membrane. It is preferred that the filter medium does not contain an adhesive layer or adhesive pastes which could cause blockage of the pore or gaps for the textile or membrane.

If the substrate 10 is a non-woven material, the non-woven material may be a sheet structure of continuous filament polyester or polypropylene fibres that are randomly arranged, highly dispersed, and bonded at the filament junctions. Meltblown materials may also be used to form the substrate 10, or part thereof. The chemical and thermal properties of spunbonded polyester are essentially those of polyester fibre. The fibres' spunbonded structure offers a combination of physical properties, such as, high tensile and tear strength, non-ravelling edges, excellent dimensional stability, no media migration, good chemical resistance, and controlled arrestance and permeability. Spunbonded polyester or polypropylene fabrics are used in various industries as covers (e.g., medical gowns or masks) or support materials. These may also be utilised in the medical industry and also for other personal protective equipment or disposable products.

Spunbonded polyester or polypropylene fabrics include either straight or crimped or polypropylene polyester fibres which give the fabrics different filtration and other general performance properties. It is believed that crimped fibres offer properties of softness, conformability, and greater porosity, while straight fibres yield stiffness, tighter structure, and finer arrestance.

The pathogen inhibiting layer of the present invention may provide for a surface that can reduce activity of pathogens and thereby reduce the residence time of potentially dangerous pathogens on surfaces. For example, the Sars-COV-2 virus has been shown to persist on some surfaces for several days, when exposed the treated textile, its persistence may be reduced to between 5 to 60 minutes. It is preferred that up to 99.9% of pathogens exposed to the surface are deactivated after a period of 60 minutes.

Optionally, the substrate 10 may be formed with at least one antibacterial or anti-pathogen chemical or nanoparticle within a fibre structure if the substrate comprises fibres. For example, a fibre of the substrate 10 may include silver or copper nanoparticles which may also release ions in addition to the pathogen inhibiting layer.

In one embodiment of the invention, nanoparticles are prepared by a process which includes PVD or CVD methods or a mixture thereof, with the vapour condensing onto a substrate to form a film with nanoparticles of the condensed vapour appearing at the surface of the film. Nanoparticles may form at least a part of a continuous coating or film which can conform to the general surface topography of the substrate 10. The nanoparticles may be protected, covered, or have a functional coating applied thereto after deposition which can assist with reducing nanoparticles from becoming dislodged from the substrate 10. Properties of functional coatings may include at least one of; flame retardant, UV absorbing, self-cleaning, hydrophobic, hydrophilic, and/or antibacterial. Other functionalisations may also be applied as is known in the art.

The reduction and/or prevention of virus transmission may be defined as a reduction of infectious viral titre of a virus of known concentration by at least 99.9% after exposure to the treated fabric. Preferably the reduction of infectious viral titre is at least 99.9%, 99.99% or 99.999%. Reduction and/or prevention of virus transmission is demonstrated by the inactivation of virus after exposure of the virus to the treated textile.

In other embodiments, the nanoparticles may suitably be formulated in an appropriate carrier, coating or solvent such as water, methanol, ethanol, acetone, water soluble polymer adhesives, such as polyvinyl acetate (PVA), epoxy resin, polyesters etc, as well as coupling agents, antistatic agents. Solutions of biological materials may also be used such as phosphate buffered saline (PBS), or simulated biological fluid (SBF). The concentration of the nanoparticles in the solution may in the range of from 0.001% (wt) to about 20% (wt). These nanoparticles may then form a coating which can be applied to the substrate 10.

In yet another embodiment, the article may comprise more than one pathogen inhibiting layer which can release ions to inhibit pathogens. It may be advantageous to allow for multiple pathogen inhibiting ions to be present at a surface to more effectively inhibit a pathogen.

In this general context, the wide-range control and modification of the metal film nano-morphology is crucial in the exploitation of their properties for desired applications, and this can be as result of the control of the deposition process parameters. One of the prime concerns towards the reliability of these technologies is the adhesion strength of the metal deposition, which can be used to create an adhesiveless film, to the substrate.

Adhesion is a characteristic property of the vapour condensing onto a substrate which can provide for atomic bonding and therefore removal of the need for an adhesive to be used to fix a pathogen inhibiting layer and a substrate 10. While it is noted that adhesion is dependent on the mechanical properties of the two materials brought into contact, the use of a vapour deposition may provide for at least a weak bond which can be encapsulated or protected by a further coating or treatment. The bonding may also be impacted by the relative contact angle of the vapour to the substrate and the temperature of the vapour and condensation rate.

In one embodiment, at high deposition rates, adatoms on the substrate surface may be buried under impinging atoms such that there is a limitation of the adatoms joining rate to other diffusing adatoms, but becoming potential nucleation sites for the arriving atoms from the vapor-phase. In contrast, when the deposition rates are low, the adatoms can probe a very high surface area per unit time, leading to a high nucleation rate at surface defects and lead to a high joining rate for diffusing adatoms. It is evident that, continuing the deposition, these two extreme situations will lead to films with different nanoscale structure and morphology.

The overall parameters determining the adatoms mobility on the substrate surface can be summarized as the adatom-surface interaction, the substrate temperature, the atoms deposition rate from the vapor-phase, and the energy of the atoms arriving on the surface from the vapor-phase. All these kinetics and thermodynamics parameters concur in establishing the film growth characteristics and, so, the final film nanoscale morphology. The effect of the adatom-surface interaction on the adatoms mobility may also have an impact on the surface topography or morphology of a coating, and may also control diffusion of ions from the coating.

Optionally, more than one coating of the same material can be made to a substrate 10 with the first coating being applied to the substrate upper surface and the second coating being applied to the upper surface 35 of the first coating 30. Alternatively, the first and second coatings can be applied to the upper and lower surfaces of the substrate. It is clear that in these two extreme cases, continuing the deposition, films of the same material will be formed but with very different nanoscale structure and morphology and, as a consequence, with very different properties such as density, adhesion behaviour, etc. Optionally, pathogen inhibiting layers may be disposed under a self-cleaning layer such that the surface of the article 1 can self-clean (such as remove oil stains) and also inhibit pathogens which contact the article surface.

For example, a self-cleaning TiO₂ layer may be applied over a pathogen inhibiting layer such as a layer containing copper or silver ions. The ions from the silver or copper layer may diffuse to the upper surface of the self-cleaning layer and promote an adverse environment for bacteria, microorganisms, viruses or other biological matter.

It will also be appreciated that the deposited coating thickness, crystallographic phase, and surface/interface morphology may impact other mechanical properties. The deposition coating may improve the reliability of electronic, optical, magnetic devices and have more than one function. An interface will be present between any two layers or coatings or combination thereof.

The grain size of the condensed deposition material may be increased or decreased by the temperature of the coating drum, the boat size, the boat temperature, the distance to the target substrate and the pressure within the chamber. It is preferred that each of these parameters are controlled during the deposition process. Further, the purity of the coating material may also affect the grain size and the formation of a desirable grain structure.

In one embodiment, the grain size of the deposition may be increased by increasing the temperature. Preferably, increasing the thermal exposure of the substrate and the rate of cooling can be advantageous to promote a larger grain size to be condensed on the substrate. This may be due to the increases of surface energy at high temperature. The grain growth mechanism is due to the transfer of atoms at higher temperature have sufficient diffusion activation energy to occupy the crystal lattice and induced the small grains by grain boundary diffusion thus the grains form in larger size. This may have a number of significant benefits in relation to increasing surface area of the pathogen inhibiting layer while also reducing the number of peaks 80 which form, which could accelerate diffusion of ions in some embodiments. It is preferred that the film formed on the surface of the substrate is at least one of a Stranski-Krastanow mode or a Volmer-Weber mode film. Both growth modes may have advantages for pathogen inhibition purposes. Again, it will be appreciated that the formation of these films may also be influenced by the condensation temperature and any annealing processes applied to the films formed.

Stranski-Krastanov growth modes form a continuous layer with peaks at the surface of the deposition. In this growth mode the adsorbate-surface interactions are generally stronger than adsorbate-adsorbate interactions which causes a continuous film to be developed with the peaks forming a surface texture or surface roughness.

In contrast to the Stranski—Krastanov growth mode, Volmer-Weber growth modes form discrete deposition locations or isolated deposition locations. In this growth mode the adsorbate-adsorbate interactions are stronger than adsorbate-surface interactions, which can form the discrete deposition locations.

In one embodiment, the film formed by a deposition may be annealed to modify the surface texture or impart a desired surface texture to the film. Optionally, quenching an annealed film may also change the properties of the film deposited and may also impart a desired surface texture. The imparting a desired surface texture may include a modification or change to the overall surface roughness of the film formed on a substrate.

Increasing the thermal exposure of the substrate 10 at the time of deposition and the rate of cooling may therefore impact the overall surface roughness of the deposition and can impact phase change of the deposition. In one example, low surface roughness of copper oxide can be obtained by high thermal exposure. Conversely, it can be expected that an increase in surface roughness can be achieved at lower thermal exposure. For example, a higher surface roughness may be achieved by annealing at around 250° C. compared to that of annealing at a temperature of 450° C. when annealing is a post process applied to the deposition. Increasing or decreasing surface roughness may have a number of benefits.

Increasing surface roughness may have the added benefit of increasing the overall surface area of the upper surface of the deposition. Increasing the surface area of the deposition may have a number of advantages in relation to the overall pathogen inhibiting properties of the surface.

The morphology and topography of the surface of the pathogen inhibiting layer may also provide improved benefits in relation to the diffusion of ions and the effectiveness of pathogen disruption.

The topographical features and morphological features of the surfaces of the first coating and/or the second coating may have a significant impact on the diffusion rate of ions from the pathogen inhibiting layer. Increasing the number of peaks at the upper surface of the pathogen inhibiting layer may also increase the diffusion rate. As such, forming the upper surface with a desired number of peaks which corresponds to a desired diffusion rate may assist with more effectively eliminating pathogens which interact with the article 1.

The peaks and valleys at the surface may be used to define the surface roughness of the coating. Qualitatively, the 0-100 nm range in the height scale for the bare pathogen inhibiting layer is just an indication of a higher surface roughness. Height differences between valleys and peaks will define the surface roughness with the roughness parameter quantifying the vertical spacing of a surface, neglecting the horizontal spacing. If the vertical spacing are large, the surface is rough; if they are small the surface is smooth. Relatively speaking surface roughnesses of greater than 50 nm (median) are considered rough and less than 50 nm (median) are considered to be smooth.

Isolated peaks which are in the highest 10% or highest 20% of peaks may be in the range of 10% to 50% higher than the median peak height and may allow for a faster rate of diffusion compared to other peaks. As such, these sites may be advantageously formed across a surface sporadically to allow for regions of higher diffusion relative to other regions on the surface.

The overall surface roughness or depth of the peaks to the valleys may be in the range of 20 to 30 nm. The height of the peaks may be changed depending on the temperature of the vapour, the cooling rate of the condensed vapour and the thickness of the coating. Increasing or decreasing the size of the vertical spacing of the grains of the surface can allow for increased or decreased diffusion of ions.

A peak 80 is determined to be the general maximum height of a grain structure. Each peak 80 formed preferably projects from the pathogen inhibiting layer at least 10 nm from the median upper surface height of the pathogen inhibiting layer.

In a further embodiment, the pathogen inhibiting layer may have an average of between four to six peaks formed at the surface per 100 nm×100 nm surface area (referred to herein as “unit area”). Preferably, the average number of peaks formed per 100 nm×100 nm surface area is in the range of three peaks to nine peaks 80. While other grains structures may be formed the general surface roughness may be in the range of 10 nm (−5 nm to +5 nm) to 60 nm (−30 nm to +30 nm). Larger grain structures may be associated with larger peaks 80 and therefore the number of peaks 80 per unit area will also be reduced.

As the thickness of the pathogen inhibiting layer is preferably in the range of 5 nm to 100 nm, the surface roughness will be governed by the relative vertical spacing of the peaks 80 compared with the overall thickness of the deposition.

The thickness of the first coating 30 may be in the range of 30 nm to 60 nm thick. During vapour deposition the grain sizes formed by the condensing vapour may be in the range of 5 nm to 30 nm depending on the pressure within the vacuum vapour deposition system. It will be appreciated that the vapour particles may also be increased with an increase in gas pressure as the mean free path of the metal or inorganic vapours is generally decreased due to a higher number of collisions which leads to the formation of larger particles being deposited.

It will be appreciated that the use of magnetic materials to form a pathogen inhibiting layer may exhibit superparamagnetism properties when particle sizes are less than around 20 nm in diameter. As such, the management of particle sizing may have applications which extend beyond pathogen inhibition properties or may be supplemental to said pathogen inhibiting properties.

As the surface texture of the pathogen inhibiting layer can govern the rate of diffusion of ions, the surface texture is preferably controlled by the thermal exposure during deposition, cooling speed, condensation rates and surface modification. The surface of the pathogen inhibiting layer my be roughened or smoothed based on the final application. Etching, chemically altering, pressing, fusing, heating, or otherwise changing the texture of the surface may be advantageous for a final application. It will be appreciated that any texture provided to the surface of a coating or article may be any predetermined texture or desired surface texture.

Further, as the coatings are generally nanometres to micrometres in thickness, imparting a shape or texture to a substrate may alter the shape or texture of one or more coatings at the same time.

Surface etching or surface texturing may also be used to impart a desired morphology or topography to the first coating. The roughening or texturing may be on either a nano or a micro scale. Chemical etching may be desirably used in some methods to alter the surface of the first coating and may also assist with priming the first coating for application of a second coating.

In addition, the structures formed on the substrate 10 and the boundaries of grains viewable with SEM, AFM and TEM equipment are relatively spherical in appearance. Spherical grains 70 may be a direct result of temperatures or nanoparticle sizes and the rate of condensation.

Each of the grains 70 are generally in the range of 40 nm to 60 nm in diameter, however while there are some homogenous regions of the surface, the surface may also comprise at least one region with larger grain structures in the range of 60 nm to 200 nm it will be appreciated that smaller grains 70 may assist with improving pathogen inhibiting properties. However, there may be instances in which a larger grain 70 size is used to form a pathogen inhibiting layer.

Preferably the peaks 80 of the surface of the deposition outnumber the corresponding valleys 90 which are viewable at the surface of the pathogen inhibiting layer. The structures and formations which define the surface topography may optionally be melted, etched or altered by a further surface altering process to more readily achieve a desired surface topography, or create a more uniform desired surface topography or morphology.

While the grain boundaries may be surrounded by a gap or pore, portions adjacent grain boundaries may abut or otherwise interact. The gaps or pores between grain boundaries are on average less than 10 nm wide but may extend for up to 50 nm in length. As such, the grain structure may have gaps or pores which are on average less than 15 nm in width and are less than 60 nm in length. However, where larger grain structures are formed the gaps between grains may be as large as 30 nm in width and 150 nm in length. The larger peaks may also provide for a faster rate of ion diffusion locally relative to smaller peaks.

It is preferred that the first coating is a continuous layer of pathogen inhibiting material which provides for a larger surface coverage compared to conventional pathogen inhibiting coatings. It will be appreciated that standard pathogen inhibiting coatings generally have less than 20% by weight pathogen disruptive material embedded or encapsulated in a binder or matrix of a gel or polymeric material. In contrast the present pathogen inhibiting coatings may be formed with at 50% pathogen disruptive material, or more preferably, greater than 60%, or even more preferably greater than 70%, or even more preferably greater than 80%, or even more preferably greater than 90%.

Visually the surface when viewed as a 2D image from above may have a “tortoiseshell” appearance with grain boundaries 75 and valleys 90 being defined by darker regions of the image, and the lighter regions of the image define the peaks 80 at the upper surface of the coating. The grain boundaries 75 can be seen in the embodiment of FIG. 9. The size of grains at the surface may be in the nanoparticle size range, while the coating as a whole may have fused or bonded nanoparticles deposited thereon to create a generally continuous structure.

Discrete formations of the surface may be formed wherein bornhardt-like or tor-like formations can be seen at the surface of the deposition layer. These formations projection from the substrate at an angle which is formed by the substrate movement during deposition relative to the vapour cloud. Other surface topographies and morphologies can be formed by displacing the vapour cloud to a different location relative to the coating drum or curved surface during deposition.

Peaks 80 and valleys 90 formed by the deposition may be dependent on the cooling rate of the vapour condensing on the substrate. Further, the surface topography may be impacted in regions by the fibres or the structure of the upper surface of the substrate 10, which may be more prominent for textiles and fabrics. As such, gaps or apertures may be formed which generally correspond to gaps, apertures, fibre spacing or other features of the substrate to be coated. It is preferred that the pathogen inhibiting layer is no greater than 300 nm in thickness to allow for minimal impact to breathability, flexibility or other mechanical properties of the substrate 10.

As the drum rotates and cools the substrate, the drum will impart a changing orientation to the substrate. The changing orientation may impart a slanted or angled deposition near to the surface. This angled deposition may be controlled by displacing the evaporation cloud relative to a curved surface which the substrate will pass over during deposition processing. The vapour density of the vapour cloud will may also impart a desired thickness or impart a desired surface texture to the film deposited. In other embodiments the substrate travels across a linear surface during deposition to impart a different surface morphology or topography of the deposition coating. The angle of the projections forming the peaks may be controlled by displacing the vapour source relative to a substrate 10 during deposition, altering the radius of curvature of a substrate 10 during deposition, or by changing the movement speed of the substrate 10.

A plurality of discrete surface structures 80, 85, 90, 95 can be formed which have a generally homogenous angled structure which may be governed by the velocity of the textile during coating. Preferably, the coating of the substrate 10 is within the range of 0.1 m/s to 10 m/s. More preferably, the speed of the textile during coating is in the range of 0.5 m/s to 5 m/s. In some embodiments, the substrate 10 is transported through the system between 0.5 m/s to 2 m/s.

A large benefit of the pathogen inhibiting layer of the present invention is that the pathogen inhibiting layer can be applied without adhesives and without reducing the porosity or the breathability of the substrate 10. Another clear advantage of the present substrate 10 and pathogen inhibiting layer is that the pathogen inhibiting layer includes a relatively higher portion of nanoparticle sized grains 70 which can increase the overall surface area of said pathogen inhibiting layer and thereby increase the potential for pathogen inhibition or destruction.

At least one surface of the substrate 10 is preferably covered at least 80% by the deposition process. It will be appreciated that the coverage of the substrate 10 may be governed by the size of fibres, structure of the fibres and the pores present in the substrate 10. As such, the deposition may appear to be entirely covered when viewed from the top, but minor changes in the deposition thickness and/or colour may be observed at angles nearer to the plane of the substrate 10. However, the deposition will still preferably form a film layer on a substrate, which is preferable a continuous film layer or a semi-continuous film layer.

The article 1 of the present invention may also have utility as an air or water filter medium. These filters may be used to purify fluids or capture undesired contaminants in fluids. The filter medium is preferably formed from a substrate with at least one of a pathogen-retaining medium and a pathogen inhibiting layer. Optionally, the pathogen inhibiting layer in this embodiment may be a membrane or is deposited onto a membrane which is used in the filter medium. Optionally, the membrane may be an article 1 comprising a substrate and at least one coating formed with a vapour deposition process.

In another embodiment, the article 1 may be a protective barrier such as a gown, a wall covering, drape, a curtain, a sheet, or another substrate used for creating a barrier to an environment or barrier to reduce or inhibit penetration of a fluid or a particle in a fluid, such as a pathogen. A pathogen retaining medium may be used to capture pathogens by either providing a physical barrier or providing a static charge which can attract and capture pathogens. This is due to pathogens having a charge which can be attracted by a statically charged substrate. At least one pathogen retaining medium may be included within the article 1 and a coating, which can be a pathogen inhibiting layer, may also be included to allow for capture and then inhibition or destruction of pathogens by the article 1. It will be appreciated that the protective barrier and the filter medium may be constructed from the same substrates and coatings. As such, the references herein to the term “filter medium” may also refer to “protective barrier”. Protective barriers may include a membrane, which may be selected from the following membranes; such as a microporous membrane, a PU membrane, a TPU membrane, a PP membrane, a PE membrane, a PET membrane, PTFE membrane, ePTFE membrane, or any other desired membrane. Protective barriers may be woven or non-woven substrates 10.

The filter medium may further comprise a dirt or chemical holding filter medium for capturing larger particles before the larger particles interact with a pathogen inhibiting layer to reduce potential biological fouling of the filter. The term “biological fouling” herein means accumulation of microorganisms on surfaces or pores of the pathogen inhibiting layer or another coating associated with the filter medium.

In another embodiment, there may be provided a filter medium (not shown) containing at least two membranes with or without pathogen inhibiting layers. The membranes may also be suitable for limiting the flow of fluids. Each membrane may be bonded with a substrate 10 or a pathogen inhibiting layer. Adhesives for fixing substrates and membranes may reduce the mechanical properties of the membrane and therefore may be disadvantageous to use. However, thermally bonding the membrane and a substrate 10 together may overcome these drawbacks. Additionally, utilisation of a nonwoven intermediate layer (not shown) may be used as a bonding layer to reduce adverse impacts on the membrane of the filter medium.

Optionally, the filter medium may restrict fluid flow in a first direction and promote fluid flow in a second direction. In this way filter mediums may be bidirectional filter mediums which can be inserted into conventional filter cartridges or other filter holding devices. For example, the filter membrane may have utility as a water purification filter medium or a filter medium which can be mounted within an air-conditioning unit. Other applications for the filter medium may include respiratory devices, masks, a water storage tank, a pump, a supply line, a water-purification device furniture textiles, flooring foundation, geotextiles, or other applications where filtration and pathogen inhibition are desired.

In one embodiment, the filter medium may be an air filter. An air filter may be used to remove contaminants, often solid particles, from air. Air filters are often used in diving air compressors, ventilation systems and any other situation in which air quality is important, such as in air-conditioning units. An air filter includes devices which filter air in an enclosed space such as a building or a room, as well as apparatus or chambers for handling viral materials. Other articles which perform a protective function such as curtains or screens may therefore also be considered as air filters. An air filter according to this aspect of the invention may therefore also be prepared according to the second aspect of the invention.

Air filters may be composed of paper, foam, cotton filters, or spun fibreglass filter elements. Alternatively, the air filter may use fibres or elements with a static electric charge. There are four main types of mechanical air filters: paper, foam, synthetics and cotton. Any desired substrate of the article 1 may be charged with either a positive or negative charge. As most viruses are generally negatively charged, the substrates may be positively charged such that the substrates, or fibres thereof, can attract viruses and capture viruses to be inhibited or destroyed by ions of a pathogen inhibiting layer. This is also advantageous as charged fibres can allow for a more open, and therefore more breathable, substrate to be formed which can capture particles, including viruses, with more than just physical means.

In another embodiment two or more substrates 10 which may be laminated or bonded together, such as the article 1 illustrated in FIGS. 15A and 15B. Each respective substrate may have a unique construction before lamination. The resulting article 1 may be a construction which is suitable for medical filtration applications, such as for use in gowns, surgical masks, curtains or the like. The lamination of multiple layers of substrates (with or without coatings) can be used for a number of applications and may allow for inclusion of multiple pathogen inhibiting layers of different structures and compositions. More than two substrates can be laminated to achieve a higher filtration performance or an improved disinfection performance It will be appreciated that the term “disinfection” herein refers to the cleaning or removal of a pathogen from a surface by inhibiting, capturing, killing or otherwise destroying said pathogen. Disinfection may take seconds to hours depending on the pathogen disruptive layer properties and the age or surface topography of the pathogen disruptive layer.

In another embodiment, the article 1 may be a barrier which includes a one or more substrates 10 and at least one coating on a substrate 10. Each respective substrate 10 may have a unique construction which can be used for any desired functional purpose, such as hydrophobicity, hydrophilicity, statically charged, pathogen inhibiting or any other predetermined function. Similar to laminated articles mentioned above, the article 1 may be a construction which is suitable for barrier applications which may include for use in gowns, surgical masks, curtains, or the like. Multiple substrates, which may each respectively have one or more respective coatings thereon, can be used for a number of applications, such as forming a protective barrier. Protective barriers may be used for a number of applications such as use for; gowns, curtains, bedding, or used for creating any other desired barrier to an environment. More than one substrate may be used to impart a desired filtration, or improve the filtration performance or improve the disinfection performance of the article 1.

Accordingly, the invention provides more efficient disinfection filter medium for air or liquid filtration. The filter may be formed to provide any desired property, such as a low pressure drop and a high flow rate when in use. It is preferred that the substrate may have an air permeability that allows a differential pressure of less than 4 mm H₂O/cm² through the substrate. Preferably, any filter material is treated with a pathogen disruptive layer. While the textile may comprise at least one pathogen disruptive layer, any number of pathogen disruptive layers can be used. Each of the pathogen disruptive layers may be formed from the same material or same pathogen inhibiting or pathogen killing material. A stacked arrangement or stacked configuration may be used with the textile which can be used to kill, filter, capture, reduce movement, inhibit, disrupt or otherwise intervene with the progression of a pathogen into the respiratory system of a person.

The use of nanoparticles may be disadvantageous for a number of applications as the bonding energy between a substrate and a nanoparticle applied through conventional methods is relatively weak, and therefore leaching can occur in use. Leaching silver or other inorganic nanoparticles can have a number of problems and environmental impacts or health impacts for a wearer. For example, silver leaching into water systems can increase algal bloom and cause ecosystem imbalances, or the consumption of silver can cause silver poising (argyria) which can cause permanent discolouration of the skin. As such, the application of silver utilising vapour deposition processes can be used to increase the overall bonding strength between a substrate and the nanoparticles deposited by the vapour relative to solution dipping or padding methods or thermal bonding methods known in the art.

The filter medium may be prepared from any suitable natural or artificial material as described above in relation to the second aspect of the invention. It is preferred that filters are formed from generally porous materials which can capture particles of any predetermined size.

Polyester fibre can be used to make web formations used for filtration devices and filter mediums. polypropylene or a polyester blended with cotton may be used to produce the filter medium. Other fibres may also be substituted for the cotton in the blended article 1. Tiny synthetic fibres known as micro-fibres may be used in many types of HEPA (High Efficiency Particulate Air Filter) filters. High performance air filters may use oiled layers of cotton gauze.

Alternatively, the filter may be used to filter liquids. Such filters may be composed of any suitable fibre as described above. Filters used to filter liquids may be used to filter potable liquids for human or animal consumption, water for general domestic use, fluids for medical use, such as plasma or saline solutions, or pharmaceutical formulations for injection, or other biological liquids which may come into contact with a patient.

According to another embodiment, there may be provided an article of protective clothing composed of fibres in which said fibres are coated with a composition of nanoparticles as defined above. The personal protective clothing may be any item of clothing which can utilise the article 1 of the present invention or benefit from a coating or treatment which is applied with a vapour deposition process to form a pathogen inhibiting layer. For example, the personal protective clothing may be a face mask. Such masks may cover the whole face of the user or a part thereof, suitably the external areas of the nose and/or mouth of the wearer.

In a preferred embodiment of the present invention there is provided a face mask or a filter composed of a fibrous non-woven material which has been coated with a pathogen inhibiting layer via a vapour deposition method. The pathogen inhibiting layer may be a composite material with one or more layers bonded or fixed together to form an article 1. The article 1 may form at least a portion of the mask or filter. Optionally, a gel, cream or other solution can be used to suspend or retain pathogen inhibiting ions (such as nanoparticles of silver or copper) which can be used to kill or reduce activity of at least one pathogen. For example, the use of mixed nanoparticles of zinc oxide (ZnO) and titanium dioxide (TiO₂) for reducing and/or preventing virus transmission. Such mixed nanoparticles of the invention may also be used in methods as described above, or in filters as described above, or articles of protective clothing as described above.

In yet a further embodiment, there is provided a method of preparing a filter. The filter may be used for at least one of; air filtration and water filtration. The method comprises thermally bonding silver or copper coated substrates 10 with a thermal bonding layer, optionally including a pathogen-retaining medium such as a non-woven material. Thermal bonding may be conducted through at least one of the following processes selected from the following group; calendaring, belt calendaring, through-air thermal bonding, ultrasonic bonding, heat bonding, lamination, and autoclave processes.

If the article 1 is to be used to form a garment, the garment may be selected from the group consisting of face masks (surgical masks, respirator masks), hats, hoods, trousers, shirts, gloves, skirts, boilersuits, surgical gowns (scrubs) etc. Such clothing may find particular use in a hospital where control of infection is important.

Referring to FIG. 14A there is shown an embodiment of an article 1 which includes a substrate 10 with a first coating 30. The article has been wound into a roll 100 which comprises a plurality of windings of the article 1. The roll 100 can be converted into further products, such as personal protective equipment, in a further manufacturing process. For example, the article 1 may be used to manufacture masks, such as surgical masks, respirators, filters, meshes, antiviral barriers, and antibacterial barriers.

The roll 100 as illustrated shows a selective coating on portions of the substrate 10. The middle of the roll 100 is treated with said selective coating, which may be a first coating 30, and the outer regions of the substrate 10 remain uncoated by said selective coating. The selective coating will be referred to herein as a first coating 30, but will be appreciated that any number of coatings may be applied to the substrate and the selective coating may be the number of coatings plus one further coating. Laminations may be applied to a substrate, or multiple coatings may be applied to the substrate 10. The roll of FIG. 14A may be a roll 100′ formed from a larger roll 100.

It will be appreciated that the substrate 10 may receive a protective coating which covers both the first coating 30 and the uncoated region 25 of the substrate 10 in a subsequent treatment or coating step. Alternatively, only one of the uncoated region 25 and the first coating 30 may receive a protective coating. The protective coating may be applied before or after the first coating 30 is applied to the substrate, and may be applied to the entire substrate or part thereof. In one embodiment, a sacrificial layer (not shown) is applied to the substrate 10 which may be removed after the first coating 30 is applied. The sacrificial layer may be used to impart a pattern or remove a portion of the first coating 30 when the sacrificial layer is removed. The sacrificial layer may be removed by peeling, solvent, washing, radiation, or any other predetermined method for removing the sacrificial layer. Said sacrificial layer may be used to impart a sharp edge or clean edge to a portion of the first coating 30.

Referring to FIG. 14B there is shown a further embodiment of a roll 100 with a plurality of sections selectively coated with a first coating 30 and several uncoated sections 25. The roll may have a width which is greater than the product to be formed with an article 1, and may be cut into smaller rolls by conventional cutting or slitting systems. The roll 100 may be cut and wound into a number of smaller rolls 100 such as that seen in FIG. 14A for example. Each smaller roll formed from the larger roll 100 and may have different coatings applied thereto.

Further, while each of the coated sections are shown as being coated with a first coating, each of the coatings may be any predetermined coating or treatment. For example, the first coating in each of the three respective sections may be of different materials, such as copper, aluminium, and gold. Any material may be used as a coating and each of the coatings applied may be referred to as a first coating 30. Optionally, portions of the substrate 10 may already have one or more coatings, and a subsequent coating may be applied instead. In this way the uncoated regions 25 may be a first coating, and the shown first coating 30 may instead be a second coating 50. Other variations of the number of coatings are also anticipated by this disclosure, and the embodiment shown is to indicate the masking or selective coating of a portion of a roll 100.

Selective coating may be applied by selectively activating evaporation boats when using an evaporative method, or may be selectively targeted by coating equipment. In yet another embodiment, the substrate may be shielded to apply the selective coatings. Shields (not shown) may be installed within the system to apply the coating to the substrate 10 and mounted to block line of sight to portions of the substrate. In this way the shields may reduce or restrict coating of the substrate 10 and cause the uncoated regions 25.

Turning to FIG. 14C there is shown an embodiment of an article 1, which is substrate 10 with a first coating 30 applied thereto. In this configuration the article 1 may be suited to be converted into a product such as a face mask 150, or another product which may require bonding, welding or adhesive to be applied. The uncoated regions 25 may be left uncoated or free from the first coating 30 such that said uncoated regions 25 may be fixed together, or folded over and bonded on itself. One or more uncoated regions 25 may be bonded together. This may be advantageous as coatings applied with PVD processes may not adhere well with another surface with a similar or the same coating. This may be particularly true when a substrate 10 with a first coating 30 is to be bonded or fixed with itself, or bonded or fixed with a second surface, such as a coating or substrate 10. Said substrate 10 with the first coating 30 may be fixed to itself by folding the substrate and bonding, or being fixed with a second substrate. As such, the selective coating of the substrate 10 may allow for regions which can be bonded more effectively compared to regions which comprise a coating applied thereto. This may be particularly the case for non-woven substrates, meltblown substrates, synthetic substrate, or any other woven substrate.

FIGS. 15A and 15B show cross-sections of an assembly including an article 1 which can be used to form a mask 150. The mask 150 may be a surgical mask or filtration mask which may be adapted for use as a barrier to protect a wearer from particles, dust, a virus, bacteria or a microorganism. If the article 1 is a mask it may be preferred that the first coating is a coating which may inhibit or destroy a virus or microorganism.

The construction of the mask 150 may include one or more layers which includes at least one article 1. As shown, the construction includes an article 1, a middle layer 165 and inner layer 160. The inner layer 160 may be the layer which is adapted to be in contact with the face of a wearer in the case of a mask 150. The inner layer 160 may be a filter medium or a membrane, and the article is a substrate 10 with at least a first coating 30 applied thereto. The construction may optionally include multiple layers which are formed from articles 1. In the illustrated embodiments, the inner layer 160 may be a spunbond non-woven substrate, the middle layer 165 may be a meltblown layer, and the article may be formed with a substrate which is spunbond substrate. A construction of layers in this order (spunbond-meltblown-spunbond) may be known within the art to manufacture surgical masks. Optionally, one or more membranes may be disposed between any of the layers 1, 165, 160 of the mask 150.

As seen in FIG. 15A there is shown an embodiment of an article 1 within a multi-layer construction. The multi-layer construction may be suitable for use as a mask 150 or for another medical device or filtration device. However, specific reference will be made to FIG. 15A as being a mask. There is illustrates 3 layers of substrate which are disposed in a stacked configuration. The upper-most layer is an article 1 formed with a substrate and at least one coating thereon, such as a first coating 30.

FIG. 15A shows an example of an unbonded construction wherein the bonding regions 180 have not been welded, fixed, adhered or otherwise stuck together. Conventional fixing processed may be used to bond the bonding regions 180 of the mask 150. The bonding regions may be formed by folding the edge of the construction over itself to form a flap 190, such that a flap bonding surface 195 can be bonded with the upper surface 27 of the uncoated region 25. It will be appreciated that by forming the flap 190, the uncoated region 25 can be bonded to the flap bonding surface 195.

As can be seen the first coating 30 may extend under the flap bonding surface 195 and be at least partially covered after bonding. This may allow for a seamless transition and give the impression that the first coating 30 extends and continues under the bonded region 180.

FIG. 15B there is illustrated another embodiment of an article 1 which is incorporated into a mask 150. The article 1 of the mask 150 has a first coating 30 which is configured such that the first coating is not overlapped by the flap 190 and the bonding at the bonding region 180 may be more effective as the first coating 30 is wholly excluded from the bonding region 180. Having the interface between the flap and the flap bonding surface 195 free from a first coating 30 may improve the bonding from ultrasonic welding thermal bonding, melting, or adhesive bonding methods if the first coating 30 comprises a metal, metal alloy, metal salt, or metal oxide.

Unlike FIG. 15B the first coating 30 does not extend under the flap 190 and instead finishes generally proximal the flap end 197. This may improve the bonding between the upper surface 27 and the flap bonding surface 195 at the bonding region 180. Further, the reduction of the surface area of the coating 30 may reduce the overall coating material required to coat a roll 100.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A substrate with a pathogen inhibiting treatment, the substrate comprising;
a first coating of an inorganic material applied to the substrate via a vapour deposition process;
a second coating at an upper surface of the first coating; and
wherein the second coating is at least one of a protective coating for the first coating and a functional coating.

2. The substrate as claimed in claim 1, wherein the inorganic material is selected from the following group; titanium, aluminium, zinc, gold, silver, cesium, copper, sulfates of calcium, strontium, barium, zinc sulfide, copper sulfide, titanium dioxide and barium zeolites, brass, mica, talc, kaolin, mullite, lead, mercury, silica and oxides of any of the preceding inorganic materials.

3. The substrate as claimed in claim 1, wherein the first coating includes pathogen disruptive ion releasing material.

4. The substrate as claimed in claim 1, wherein the second coating comprises one or more pores to allow the diffusion of ions from the first coating to an exposed surface of the second coating.

5. The substrate as claimed in claim 1, wherein an average surface roughness of the upper surface of the first coating is in the range of 10 nm to 30 nm.

6. The substrate as claimed in claim 1, wherein the average surface roughness of the second coating is in the range of 10 nm to 50 nm.

7. The substrate as claimed in claim 1, wherein the first coating comprises a surface morphology in which grain peaks project from the surface of the substrate at an angle of between 20 degrees to 40 degrees relative to perpendicular angle of the surface.

8. The substrate as claimed in claim 1, wherein the first coating is a continuous coating of pathogen inhibiting material across at least 80% of the surface of the substrate.

9. The substrate as claimed in claim 1, wherein an interface between the first coating and the second coating allows for diffusion of ions from the first coating through to an upper surface of the second coating.

10. The substrate as claimed in claim 1, wherein the average diameter of at least 80% of the grains of the first coating at the upper surface is in the range of 5 nm to 50 nm.

11. The substrate as claimed in claim 1, wherein the substrate has a differential pressure of less than 4 mm H₂O/cm².

12. An article with a pathogen inhibiting coating, the article comprising;
a non-woven substrate;
a first coating applied to the non-woven substrate, in which the first coating is a pathogen inhibiting layer; and
wherein the pathogen inhibiting layer comprises an inorganic material which forms at least 80% by weight of said pathogen inhibiting layer and is adapted to release ions to inhibit a pathogen.

13. The article as claimed in claim 12, wherein a second coating is applied to the article.

14. The article as claimed in claim 13, wherein the second coating is applied to the first coating.

15. The article as claimed in claim 12, wherein the inorganic material is selected from the following group; titanium, aluminium, zinc, gold, silver, cesium, copper, sulfates of calcium, strontium, barium, zinc sulfide, copper sulfide, titanium dioxide and barium zeolites, brass, mica, talc, kaolin, mullite, lead, mercury, silica and oxides of any of the preceding inorganic materials.

16. The article as claimed in claim 12, wherein the first coating has a surface roughness in the range of 5 nm to 50 nm.

17. The article as claimed in claim 12, wherein at least one of the substrate and the first coating are statically charged.

18. The article as claimed in claim 12, wherein the first coating is formed by a Stranski-Krastanow growth mode.
